(19) United States
(12) Patent Application Publication
(10) Pub. No.: US 2007/0178060 A1
(43) Pub. Date: Aug. 2, 2007

(54) Fresh body care products and system

(76) Inventor: Rulin Xiu, Pahoa, HI (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/341,670

(22) Filed: Jan. 30, 2006

(51) Int. Cl. A61K 36/18; A61K 8/97

(52) U.S. Cl. 424/74; 424/725

(57) ABSTRACT

The present invention relates to body care products that are comprised of fresh ingredients. It has been found that fresh ingredients, such as fruits, vegetables, and natural herbs, when applied to the skin and hair, improve texture, tone, and other features. The products can be stored in containers and compartments, e.g., that extend their shelf-life.

The present invention relates to body care products that are comprised of fresh ingredients. It has been found that fresh ingredients, such as fruits, vegetables, and natural herbs, when applied to the skin and hair, improve texture, tone, and other features, in marked improvement to body care products that contain processed or synthetic ingredients. The body care products can be packaged into dispensers which are effective to extend the shelf-life of the perishable and fresh ingredients. The present invention also provides for methods of blending ingredients from pre-packaged individual compartments.

In one embodiment, the present invention provides a body care product which comprises a plurality of perishable fruits or vegetables which are effective for improving a feature of the skin or hair.

Any fruit or vegetable that is effective in improving skin or hair vitality can be utilized in accordance with the present invention. For example, natural products that contain alpha-hydroxy, beta-hydroxy, and other naturally occurring acids can be utilized. Other useful properties include, e.g., antioxidants; exfoliants; vitamins; minerals; skin permeation enhances; moisturizers; anti-inflammatory; cell stimulants; etc.

Examples of fruits and vegetables include, e.g., apple (e.g., as a source of alpha hydroxy acid); pear; apricot; banana (e.g., can improve skin dryness); melons; cantaloupe (e.g., can improve skin's ability to retain moisture; grape (e.g., as an antioxidant and to improve moisture); mango; citrus, such as orange, lemon, lime, and grapefruit; pineapple (e.g., as an exfoliate and/or to reduce inflammation); strawberry (e.g., as a source of alpha hydroxy acid; to even skin tone); watermelon; kiwi fruit; avocado; papaya; celery; cucumber; radish; peach (e.g., contain acids as an exfoliant); carrot; beet; tomato; mint; taro; bread fruit; sea weeds; yam; sweet potato; mungbean; soy bean; sesame seeds; wheat; passion fruit; logan; lychee; etc.

Any part of a fruit or vegetable can be utilized, including root, stem, leaf, fleshy material, fruit, seed, seedpod, bud, flower (including flower petals), bark, skin, rind, phloem, xylem, sap, syrup, etc. For example, the leaf of certain plants, such as aloe vera contain a gel which can utilized in a body care product of the present invention. These parts can be used in ripe or an unripened form.

Other ingredients can be included as well, including herbs (dry or fresh); extracts of herbs; nuts; essential oils (such as Angelica Root; Anise; Beeswax; Bergamot Mint; Carrot Seed; Chamomile; Citronella; Eucalyptus; Geranium; Ginger; Grapefruit; Jasmine; Lavender; Lemongrass; Mandarin; Myrtle; Orange; Peppermint; Rose; Rosemary; Rosewood; Tea Tree; Vanilla); olive oil and other vegetable oils; sugar (e.g., brown sugar); honey; beans; grains; sea wheat; natural minerals such as sea salt; pearl powder; etc.

The body care products in accordance with present invention are “perishable.” By the latter term, it meant that they are “fresh” and have not been subjected to chemical processing, or chemical treatments, and have not undergone substantial decay or rotting. Generally, a fresh product is delivered to the consumer within about 1-2 weeks of being treated as described below for inclusion into a body care product of the present invention. The body care products can also preferably exclude exogenously added preservatives, such as sulfur dioxide, sulfites, benzoates, etc. However, natural ingredients can be utilized as preservatives, including, e.g., honey and fruits (e.g., benzoate containing fruits such as cranberry).

Although such products are not subjected to chemical processing or treatment, they may be mechanically processed in order to combine them into an effective body care product. For example, ingredients utilized in the body care preparations can be combined together in any suitable way, including blending, mixing, mashing, emulsifying, beating, grinding, etc. The ingredients can be completely pureed so that no lumps are present, or the can be incompletely mashed, such that whole pieces of ingredient remain intact.

In another aspect of the present invention, the plurality of vegetables or fruits are contained in separate compartments. For example, a body care package can comprises a plurality of compartments, wherein each compartment contains at least one ingredient, which is effective for improving skin or hair texture or appearance. For example, a banana and strawberry puree can each be stored in a separate compartment from each other. Furthermore, other ingredients, such as honey, essential oils, herbal extracts, etc., can be placed into separate containers. A consumer, upon opening the package, can then blend the ingredients together and apply it as directed to the skin or hair.

Any feature of the skin or hair can be improved in accordance with the present invention. By the term “improved,” it is meant that the quality of the appearance or texture of the feature of interest is better after treatment with the body care product. Improvement can occur after one treatment, or after a plurality of treatments, since some products may require more than one application to exert their beneficial effect.

Examples of skin appearance and/or texture that can be improved, include for example, reduce frequency and depth of wrinkles; improve smoothness; even tone; reduce pore size; reduce appearance of fine wrinkles and/or lines; reduce blotchiness; reduce dryness and/or flaking; improve skin clarity; remove skin discolorations and other skin markings; promote new growth; stimulate skin growth; improve skin firmness; improve elasticity; reduce sagging; improve hydration; moisturize; remove build-up of dead skin, dirt, and/or grime; increase resistance to environmental damage, including dirt; features listed below; etc. Furthermore, the skin care products can be used to improve acne and blemishes; to promote wound healing; to promote healing; to cleanse the skin; to treat sunburn; to eliminate itching; to improve skin allergy, including redness and itching.

Examples of hair appearance and/or texture that can be improved, include for example, reduce brittleness; reduce dryness and/or scalp flaking; increase smoothness; reduce split ends; improve sheen; improve strength; improve damaged hair, e.g., damaged by chemical treatments; increase softness; reduce hair fragility; improve natural coloring and highlights; improve condition of hair follicles; reduce oiliness; reduce accumulation of airborne dust and dirt; moisturize; increase elasticity; remove build-up of dead skin, grime, and/or dirt; increase resistance to environmental damage, including dirt; reduce frizziness; features listed above; etc.

The product can be in any form which is suitable for topical application. These include, e.g., alcoholic solution, aqueous solution, lotion, cream, stick, spray, ointment, paste, mousse, colloidal preparation, emulsified preparation, dehydrated preparation, masks, gel, etc. Examples of body care products include body refresher; facial or hair cleanser; mask; shampoo; hair conditioner; soap; exfoliant; tooth paste; mouth wash; shaving cream; after-shave lotion, cream, or tonic; facial cosmetic (e.g., a cream); hair tonic; deodorant; healing salve; anti-itch; lip balm; wound cleanser; etc.

Body care products of the present invention can also be provided in a dispenser. By the latter term, it is meant the container in which the product is stored prior to application by a consumer. Various container and container materials can be utilized without limitation.

The container can be utilized to extend the shelf-life of the perishable ingredients in the product. Perishable ingredients will normally spoil after a certain period of time, depending upon the temperature and conditions under which they are stored. Bacteria and fungi normally present, or which are subsequently introduced, can grow, leading to decay and rotting. A “shelf-life” is the period in which a product can be stored without substantial spoiling. Containers of the present invention can be utilized to extend the shelf-life, i.e., increase the amount of time a product can be spoiled in comparison to the storage time without the container. Improvement can be of any value, e.g., for extending the shelf-life by a day, two days, three days, four days, five days, a week, or more. Such improvements are significant when items are being stored for commercial sale. There are various containers that be utilized to achieve this goal.

For example, certain materials have natural preservative effects, such as antibiotic, anti-bactericidal, or anti-oxidant effects. These include, e.g., wood and clays. Other suitable materials for a dispenser in accordance with the present invention, include, e.g., glass; metal; composites; coated papers, such as a wax-coated paper, including a beeswax coated paper (e.g., having natural preservative properties); treated bamboo or wood; stone; ceramic; etc.

The products can also be stored in substantially air-impermeable food packaging (e.g., in a pouch or bag, e.g., made of plastic or other suitable polymers), where the container is evacuated with a vacuum prior to sealing. Additionally, modified atmosphere packaging (MAP) can be used during packaging, altering the composition of gases in contact with the food by replacing air with a single gas or a mixture of gases, e.g., to reduce or exclude reduce oxygen present in the body care product. The container (dispenser) can have a port or other means for cutting or tearing open to extrude the contents when the consumer is ready to use.

Thermal insulation materials can also be utilized. Examples without limitation, include, fabric; silica fiberglass fabric; ceramic fiber textile; cellulose fiber; polystyrene; foam; Styrofoam; packaging films; etc. The materials can further incorporate a cooled liquid or gel, such as ice or blue ice.

The present invention also provides a body care product system, comprising at least two of the following elements, e.g., (a) a body care product of claim 1 encased in a container; (b) an expiration label on said product indicating the shelf-life of said product; and/or (c) a refrigeration system for storing the body care product. This system can be utilized to preserve products of the present invention, e.g., by allowing the shelf-life to be tracked, and by providing a refrigerant system to extend the storage time of the body care product. The system can further comprise a scanner for inputting information from a bar-coded expiration label. Labels can be bar- or otherwise coded with information such as shelf-life of the products in the body care product container; ship date; date on which the products were packaged; content of container; etc.

What we claim:

1. A body care product, comprising:
a plurality of perishable fruits or vegetables which are effective for improving skin or hair texture or tone, wherein said fruit or vegetable is fresh and has not undergone substantial decay.

2. A body care product of claim 1, with the proviso that said product does not comprise a synthetic preservative.

3. A body care product of claim 2, with the further proviso that said product does not comprise a synthetically manufactured ingredient.

4. A body care product of claim 1, which is a cream or an aqueous solution.

5. A body care product of claim 1, which further comprises an essential oil.

6. A body care product of claim 1, which further comprises an herbal extract.

7. A body care product of claim 1, wherein said plurality of vegetables or fruits are contained in separate compartments.

8. A body care product dispenser, comprising
a body care product of claim 1 which is encased in a container effective to extend the shelf-life of said perishable fruits or vegetables.

9. A body care product dispenser of claim 8, wherein said dispenser is a vacuum-sealed air-impermeable pouch.

10. A body care product dispenser of claim 8, wherein said dispenser is a thermally insulated container.

11. A body care product dispenser of claim 8, wherein said dispenser comprises an inner layer of wax-coated paper in contact with said product.

12. A body care product dispenser of claim 8, wherein said dispenser is comprised of wood, bamboo, clay, glass, stone, or a metal material.

13. A body care product of claim 1 which is for facial skin.

14. A body care product of claim 1 which is for hair.

15. A body care product system, comprising:
(a) a body care product of claim 1 encased in a container,
(b) an expiration label on said product indicating the shelf-life of said product, and
(c) a refrigeration system for storing the body care product.

16. A body care product package, comprising:
a plurality of containers, each containing at least one perishable fruit and/or vegetable for application to the skin or hair upon blending the contents thereof.
